(19) United States
(12) Patent Application Publication
(10) Pub. No.: US 2007/0231807 A1
(43) Pub. Date: Oct. 4, 2007

(54) Method for preparing short hairpin RNA from CDNA

(75) Inventor: Yin-Yuan Mo, Springfield, IL (US)

Correspondence Address: SENNIGER POWERS, ONE METROPOLITAN SQUARE, 16TH FLOOR, ST LOUIS, MO, 63102, US

(73) Assignee: Board of Trustees of Southern Illinois University, Springfield, IL (US)

(21) Appl. No.: 11/397,185

(22) Filed: Apr. 4, 2006

(51) Int. Cl. C12Q 1/68; C12P 19/34

(52) U.S. Cl. 435/6; 435/91.2

(57) ABSTRACT

The present invention provides a method for generating double stranded palindromic DNA sequences which form short hairpin RNAs in a host cell, wherein a nicking DNA enzyme opens a double stranded DNA and converts it to a single stranded palindromic structure. The present invention also provides methods for expressing shRNA in a host cell, suppressing gene expression, identifying potential short interfering RNA sequences, and identifying genes associated with a particular phenotype.

This invention was made with Government support under NIH Grant No. RO1-CA10202630 awarded by the National Institutes of Health. The Government has certain rights in the invention.

### FIELD OF THE INVENTION

The present invention relates to a method for preparing double stranded palindromic DNA sequences, which form short hairpin RNAs in a host cell from either a cDNA library or cDNA from a gene of interest. Such short hairpin RNA can be used to suppress gene expression, identify potential siRNA target sequences and study gene function.

### BACKGROUND OF THE INVENTION

RNA interference (“RNAi”) refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The process of RNAi begins by the presence of a long dsRNA in a cell, wherein the dsRNA comprises a sense RNA having a sequence homologous to the target gene mRNA and antisense RNA having a sequence complementary to the sense RNA. The presence of dsRNA stimulates the activity of a ribonuclease III enzyme referred to as Dicer. Dicer is involved in the processing of the dsRNA into short pieces of dsRNA known as short interfering RNAs (siRNAs) (Berstein et al., 2001, Nature, 409, 363). Short interfering RNAs derived from Dicer activity are typically about 21 to about 23 nucleotides in length and comprise about 19 base pair duplexes (Elbashir et al., 2001, Genes Dev., 15, 188). siRNAs in turn stimulate the RNA-induced silencing complex (RISC) by incorporating one strand of siRNA into the RISC and directing the degradation of the homologous mRNA target.

The original RNAi, which was discovered in invertebrates and employed dsRNAs with length greater than 30 nucleotides was not effective in mammalian cells. This was found to be due to the fact that long dsRNAs (greater than 30 nucleotides) elicit interferon responses, resulting in nonspecific mRNA degradation and inhibition of protein synthesis. This problem was overcome by the finding that smaller double-stranded siRNAs with the length of 20-23 nucleotides do not induce an interferon response yet remain potent and specific inhibitors of endogenous gene expression (Elbashir et al., Nature 411, 494-498, 2001).

RNAi can find application in a number of genetic processes. Considering the huge quantity of genome data currently available, a reverse genetics approach can be used to examine the role of each gene by the loss-of-function method. To that end, gene targeting using homologous recombination is widely used but is labor-intensive, which precludes its versatile and convenient application. Antisense oligonucleotides can be used instead but are often characterized by toxicity, instability and nonspecific effects. RNAi is thus a good alternative to the above two approaches. In research laboratories, two types of siRNA have been widely used to suppress exogenous as well as endogenous gene expression: synthetic siRNA and vector-based siRNA (i.e. in vivo transcribed siRNA). The vector based siRNA is usually generated through short hairpin RNA (shRNA). In this system, RNA polymerase III promoters, such as H1 promoter and U6 promoter are used to drive transcription of shRNA. The shRNA transcript consists of a 19- to 29-bp RNA stem, with the two strands joined by a tightly structured loop. shRNA is processed in the cell into siRNA through the action of the Dicer family of enzymes. Thus, the transcribed products mimic the synthetic siRNA duplexes and are as effective as the synthetic siRNA for suppressing their corresponding genes.

The siRNA-mediated gene silencing efficiency is determined by many factors, the major one being the selection of a target sequence. It is well known that not all sequences of a gene are capable of generating functional siRNA. Accordingly, there is a need for new and/or improved methods of generating siRNA from cDNA.

### BRIEF SUMMARY OF THE INVENTION

Among the aspects of the present invention is the provision of a method for preparing a double stranded palindromic DNA sequence which forms a short hairpin RNA in a host cell. The method involves:

a) obtaining a DNA oligonucleotide,

b) obtaining cDNA of a gene of interest or a cDNA library, wherein the cDNA contains a 5′ CG overhang;

c) ligating the DNA oligonucleotide with the cDNA to form a ligated product;

d) digesting the ligated product with MmeI to form an MmeI-digested product;

e) ligating the MmeI-digested product with an adaptor oligonucleotide to form a ligated MmeI-digested product;

f) treating the ligated MmeI-digested product with a nicking enzyme N.Alw I to form a nicked product;

g) removing or inactivating N.Alw I from the nicked product;

h) exposing the nicked product to an elevated temperature such that the nicked product becomes single-stranded downstream from a nick site and remains double-stranded elsewhere in the sequence, thereby generating a single-stranded palindromic sequence; and

i) generating a double-stranded palindromic DNA sequence from the single-stranded palindromic DNA sequence. The DNA oligonucleotide comprises a primer encoding a sequence which is complementary to the 3′ end of a promoter, a 5′ CG overhang, an MmeI recognition site, a restriction enzyme recognition site containing GGATCC, and an N.Alw I recognition site, wherein the MmeI recognition site overlaps the restriction enzyme recognition site, and the N.Alw I recognition site overlaps the restriction enzyme recognition site. The adaptor oligonucleotide is an oligonucleotide which forms a double-stranded stem and a loop. In one embodiment, the restriction enzyme whose recognition site is GGATCC is BamH1.

It is another aspect of the invention to provide a method for generating a double stranded palindromic DNA sequence which forms a short hairpin RNA in a host cell by digesting a DNA oligonucleotide; digesting cDNA of a gene of interest or cDNA library with at least one enzyme which creates a 5′ CG overhang; ligating the primer containing DNA fragment with the digested cDNA to form a ligated product; digesting the ligated product with MmeI to form a MmeI-digested product; ligating the MmeI-digested product with an adaptor oligonucleotide to form a ligated MmeI-digested product, wherein the adaptor oligonucleotide forms a double-stranded stem and a loop; treating the ligated MmeI-digested product with a nicking enzyme N.Alw I to form a nicked product; removing or inactivating N.Alw I from the nicked product; exposing the nicked product to an elevated temperature such that the nicked product becomes single-stranded downstream from a nick site and remains double-stranded elsewhere in the sequence, thereby generating a single-stranded palindromic sequence; generating a double-stranded palindromic DNA sequence from the single-stranded palindromic DNA sequence; digesting the double-stranded palindromic DNA sequence with BamH1 to form a BamH1-digested product, and inserting the BamH1-digested sequence into an expression vector. The DNA oligonucleotide comprises a primer encoding a sequence which is complementary to the 3′ end of H1 promoter with FauI, wherein the DNA oligonucleotide also contains an MmeI recognition site which overlaps a BamH1 recognition site, an FauI recognition site immediately after the MmeI recognition site, and an N.Alw I recognition site which overlaps the BamH1 recognition site.

Another aspect of the present invention is a method for expressing short hairpin RNA in a host cell by transfecting the host cell with a vector comprising a double stranded palindromic sequence produced as described herein, which forms the short hairpin RNA in the host cell.

It is also an aspect of the present invention to provide a method for suppressing expression of a gene of interest by transfecting a host cell with a vector comprising a double stranded palindromic sequence produced as described herein, wherein the double stranded palindromic sequence forms a short hairpin RNA in the cell and targets the gene of interest.

In another aspect, the present invention relates to a method for identifying potential short interfering RNA sequences, wherein the method comprises inserting into a host cell a vector comprising a double stranded palindromic sequence produced as described herein, determining which gene exhibits suppressed expression, and identifying a short interfering RNA sequence, i.e., the double stranded palindromic sequence which targets said gene.

It is another aspect of the present invention to provide a method of identifying at least one gene associated with a selected phenotype, wherein the method comprises transfecting a host cell which exhibits the selected phenotype with a library of expression vectors encoding short hairpin RNAs produced as described herein; and identifying, in the host cell which has lost the expression of the selected phenotype, the at least one gene whose expression is suppressed by the short hairpin RNA, wherein the at least one gene so identified is associated with the selected phenotype.

Other objects and features will be in part apparent and in part pointed out hereinafter.

### FIGURES

FIGS. 1A and 1B depict a description of molecular manipulation strategy. In step 1, preparation of an adaptor and cDNA by PCR and restriction digestions is depicted. In ligating step 2, H1 promoter adaptor was digested with FauI and then ligated to the restriction digested cDNA. After ligation, the ligated products were digested with MmeI to produce the same size cDNA (20 bases). Ligation of siRNA-loop-6 made it possible to form a palindromic structure in the next step. In step 3, generation of a hairpin structure is depicted. Digestion of the nicking enzyme N.Awl I led to the formation of a fragment at the loop region at 72° C. because the region upstream of the nick was longer than the downstream region. Extension converted the single-stranded DNA to double-stranded DNA. In the cloning step 4, the extended product was digested with BamH1 and then ligated to Pu-H1-pSK-X.

FIG. 2 depicts generation of GFP-siRNAs as shown by agarose gel analysis. In FIG. 2(A), lanes 1 and 2 show GFP cDNA after digestion with Hinp1 I and HypCH4 IV, and GFP cDNA before restriction digestion, respectively. FIG. 2(B) depicts a PCR product carrying the H1 promoter, and a PCR product carrying the H1 promoter digested with Fau I in lanes 1 and 2, respectively. In FIG. 2(C), lane 1 shows a PCR product carrying the H1 promoter digested with Fau I, and lane 2 shows the same Fau I-digested fragment, which was first ligated to the restriction-digested GFP cDNA, and then digested with Mme I. An arrow indicates ligated product carrying 20 bp GFP cDNA. In FIG. 2(D), lane 1 depicts the Mme I-digested fragment as lane 2 in FIG. 2(C); lane 2 depicts the same fragment but after ligation with siRNA-loop-6. M refers to a 25 bp DNA ladder (Invitrogen).

FIG. 3 depicts suppression of GFP expression as measured by a decrease in green fluorescence intensity by GFP-siRNA-1, GFP-siRNA positive control and the vector control.

### DEFINITIONS AND ABBREVIATIONS

A “bp” is an abbreviation for base pair.

A “ds” is an abbreviation for double-stranded.

A “GFP” is an abbreviation for green fluorescent protein.

An “nt” is an abbreviation for nucleotide.

An “shRNA” is an abbreviation for short hairpin RNA.

An “siRNA” is an abbreviation for short interfering RNA.

An “RNAi” is an abbreviation for RNA interference.

A “target gene” refers to any gene suitable for regulation of expression, including both endogenous chromosomal genes and transgenes, as well as episomal or extrachromosomal genes, mitochondrial genes, chloroplastic genes, viral genes, bacterial genes, animal genes, plant genes, protozoal genes and fungal genes.

A “library” as used herein refers to a collection of nucleic acid sequences that possesses a common characteristic. For example, a library of nucleic acids can be representative of all possible configurations of a nucleic acid sequence over a defined length. Alternatively, a nucleic acid library may be a collection of sequences that represents a particular subset of the possible sequence configurations of a nucleic acid of a defined length. A library may also represent all or part of the genetic information of a particular organism. A nucleic acid “library” is typically, but not necessarily, cloned into a vector.

A “sense siRNA strand” refers to the siRNA strand that matches the target mRNA sequence.

An “antisense siRNA strand” refers to the siRNA strand that is complementary to the target mRNA sequence and is thought to induce RNAi.

An “siRNA” refers to a nucleic acid that forms a double stranded RNA, wherein the double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. The sequence of the siRNA can correspond to the full length target gene, or a subsequence thereof.

A “palindrome” or “inverted repeat” refers to a nucleic acid sequence comprising a sense and an antisense element positioned so that they are able to form a double stranded siRNA when the repeat is transcribed. The inverted repeat may optionally include a linker or a heterologous sequence between the two elements of the repeat. The elements of the inverted repeat have a length sufficient to form a double stranded RNA.

A “pol III promoter” refers to a promoter that is recognized by RNA polymerase III to initiate transcription. Examples of pol III promoters include, e.g., human and mouse H1 promoters, the human U6, 5S, U6, adenovirus VA1, Vault, telomerase RNA, and tRNA gene promoters. In addition, several other pol III promoter elements have been reported including those responsible for the expression of Epstein-Barr-virus-encoded RNAs (EBER), and human 7SL RNA.

A “Pol III terminator” refers to a sequence recognized by RNA polymerase III to terminate transcription. Exemplary pol III terminators include four or five contiguous thymidines (e.g., TTTT or TTTTT).

The terms “substantially identical” or “substantial identity,” in the context of two or more nucleic acids or polypeptide sequences, refer to two or more sequences or subsequences that are the same or have a specified percentage of amino acid residues or nucleotides that are the same (i.e., at least about 60%, preferably 65%, 70%, 75%, more preferably 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 6-7 amino acids or 25 nucleotides in length, or more preferably over a region that is 50-100 amino acids or nucleotides in length.

The term “recombinant” when used with reference, e.g., to a cell, or nucleic acid, protein, or vector, indicates that the cell, nucleic acid, protein or vector has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

### DETAILED DESCRIPTION OF THE INVENTION

The present invention provides a method for generating short hairpin RNA (shRNA) from cDNA, and more specifically double stranded palindromic DNA sequences which form short hairpin RNAs in a host cell. In the last few years, several methods have been reported for the generation of shRNA. See, e.g., Luo et al., (PNAS, 101(15):5494-5499 (2004)), Sen et al., (Nature Genetics, 36(2):183-189 (2004)), and Shirane et al., (Nature Genetics, 36(2):190-196 (2004)). In these systems, cDNA is cleaved into small fragments by either restriction enzymes or DNase and is subsequently ligated to an artificial adaptor or loop. Through complex and multiple steps of manipulations, a ligated double-stranded cDNA is then converted into a 20-bp palindromic structure and cloned into a vector for expression in a host cell. These steps can be tedious and technologically challenging, as is the case with the conversion of a double-stranded cDNA into a palindromic structure. The present invention provides a method for preparing shRNA from cDNA, wherein the present method overcomes several limitations associated with the previous methods. More specifically, a small loop is used so that it is unnecessary to remove the loop after cloning in order to produce functional shRNAs. Furthermore, a nick enzyme (eg. N.Alw I) allows for easy opening of the stem-loop structure, and a DNA polymerase allows for easy conversion of a stem-loop structure into a palindromic structure.

Accordingly and in one embodiment, the present invention provides a method for generating a double stranded palindromic DNA sequence which forms a short hairpin shRNA in a host cell, wherein the method comprises the following steps:

a) obtaining a DNA oligonucleotide, which comprises a primer encoding a sequence which is complementary to the 3′ end of a promoter, a 5′ CG overhang, an MmeI recognition site, a restriction enzyme recognition site containing GGATCC, and an N.Alw I recognition site, wherein the MmeI recognition site overlaps the restriction enzyme recognition site, and the N.Alw I recognition site overlaps the restriction enzyme recognition site;

b) obtaining cDNA of a gene of interest or a cDNA library, wherein the cDNA contains a 5′ CG overhang;

c) ligating the DNA oligonucleotide with the cDNA to form a ligated product;

d) digesting the ligated product with MmeI to form an MmeI-digested product;

e) ligating the MmeI-digested product with an adaptor oligonucleotide to form a ligated MmeI-digested product, wherein the adaptor oligonucleotide forms a double-stranded stem and a loop;

f) treating the ligated MmeI-digested product with a nicking enzyme N.Alw I to form a nicked product;

g) removing or inactivating the nicking enzyme from the nicked product;

h) exposing the nicked product to an elevated temperature such that the nicked product becomes single-stranded downstream from a nick site and remains double-stranded elsewhere in the sequence, thereby generating a single-stranded palindromic sequence; and

i) generating a double-stranded palindromic DNA sequence from the single-stranded palindromic DNA sequence.

In one embodiment, the restriction enzyme whose recognition site is GGATCC is BamH1. Furthermore, BamH1 can be used to digest the double-stranded palindromic sequence, which can be inserted into a vector of choice. Once expressed in a host cell, the double stranded palindromic sequence is processed into a short hairpin RNA.

In the present method, a DNA oligonucleotide can either be obtained (e.g., from a commercial source which synthesizes oligonucleotides) or can be prepared as described below. Chemical synthesis of linear oligonucleotides is well known in the art and can be made by any of several different synthetic procedures including the phosphoramidite, phosphite triester, H-phosphonate and phosphotriester methods, typically by automated synthesis methods. Beaucage and Caruthers, Tetrahedron Letts., 22:1859-1862 (1981); Needham-VanDevanter et al., Nucleic Acids Res., 12:6159-6168 (1984). Moreover, oligonucleotides can also be custom-made and ordered from a variety of commercial sources known to persons of skill in the art.

A DNA oligonucleotide used in the present invention contains from about 40 to about 70 nucleotides. In one embodiment, the DNA oligonucleotide contains from about 50 to about 60 nucleotides. In still another embodiment, the DNA oligonucleotide contains about 54 nucleotides. There are several features that characterize the DNA oligonucleotide used in the present invention. Specifically, the DNA oligonucleotide comprises:
I) a primer encoding a sequence complementary to the 3′ end of promoter,
II) a 5′ CG overhang,
III) an MmeI recognition site,
IV) a restriction enzyme recognition site containing GGATCC, wherein the MmeI recognition site overlaps the restriction enzyme recognition site, and
V) an N.Alw I recognition site, wherein the N.Alw I recognition site overlaps the restriction enzyme recognition site.

In one embodiment, the restriction enzyme whose recognition site contains GGATCC is BamH1.

The primer encodes a sequence from about 9 nucleotides to about 30 nucleotides, which is complementary to the 3′ end of a promoter. The promoter is selected based on the promoter that is used to drive the expression of shRNA in the vector, which will be transfected in a host cell. By way of example, if a vector used to express shRNA in a host cell contains a U6 promoter, then the primer encodes a sequence which is complementary to the 3′ end of the U6 promoter. One skilled in the art will recognize that the choice of a promoter and vector depends on the cell type in which shRNA will be expressed, and can readily determine which promoters and/or vectors are suitable for a cell type. For example, if plant cells are to be the target, then plant promoters should be used. The promoters can be constitutive, inducible, or cell dependent, depending on the application and result desired.

The promoters used in the present invention are preferably selected from polymerase I, II and III dependent promoters. Preferably, a promoter used in the present methods is a polymerase II or III dependent promoter including, but not limited to, a CMV promoter, a CAGGS promoter, a snRNA promoter such as U6, a RNAse P RNA promoter such as H1, a tRNA promoter, a 7SL RNA promoter, a 5 S rRNA promoter, and the like. The promoter can be a constitutive promoter, or can be an inducible promoter. Suitable inducible promoters are the above-mentioned polymerase I, II and III dependent promoters containing an operator sequence including, but not limited to tet, Gal4, lac, etc.

In addition, the RNA polymerase III promoter constructs of the invention may comprise various elements to allow for tissue specific, or temporally specific expression. Methods to achieve such tissue or temporally controlled expression are known in the art and any of these may be employed to achieve such expression. By using such mechanisms this may allow the inhibition of the target gene to occur in a specific cell type or stage of development. This can have applications in both therapy and developmental biology for example, where the aberrant expression or mutated allele is only being expressed in a particular cell type or if it is not wished to disrupt expression in other cell types or where a gene is only expressed during a particular stage of embryonic development or maturation of the adult organism. Such promoters also allow for the study of essential embryonic genes in mature adults.

In one embodiment, the promoter used in the present invention is an RNA polymerase III promoter. In another embodiment, the promoter is the H1 promoter. In still another embodiment, the vector used for expression of shRNA comprises five thymidines downstream of the cloning site, wherein the thymidines act as a terminator sequence for a Pol III polymerase.

The primer also comprises recognition sites for MmeI, BamH1, N.Alw I and a restriction enzyme which generates 5′ CG overhangs, wherein MmeI recognition site overlaps BamH1 recognition site, which in turn overlaps N.Alw I recognition site. MmeI is selected because it cuts 20 nucleotides 3′ from its recognition sequence TCCRACN, generating fragments of uniform length. BamH1 site (GGATCC) overlaps MmeI site and is used for cloning purposes. It is of note that a restriction enzyme other than BamH1 can be used, as long as its recognition site overlaps the MmeI recognition site and it contains the sequence GGATC, which is a part of the recognition sequence for the nicking enzyme N.Alw I (GGATCNNNNˆN) required to open up a double stranded DNA in a later step. For example, XholI enzyme recognizes the sequence (A/G)GATC(T/C) and can be used instead of BamH1.

A primer also comprises a recognition site for the restriction enzyme which generates 5′ overhangs and is situated immediately following the MmeI recognition site. Any enzyme which generates 5′ overhangs can be used, including but not limited to FauI, HinpI, Hinp1I, BsaHI, AciI, HpalI, HpyCHIV, HpyCH4IV, TaqaI, Acl I, DspD I, Cla I, Msp I, Nar I, and BstB I. In one embodiment, the enzyme that is used to generate a 5′ overhang on the DNA oligonucleotide is FauI. In another embodiment, a stuffer sequence is included 3′ of the FauI site to increase the digestion efficacy of FauI. The stuffer sequence is a stretch of DNA, from about 5 to about 20 bp in length, which increases the digestion efficiency of the restriction enzyme, such as FauI. The digestion with FauI can be performed, e.g., by incubating 5 micrograms of DNA with 10 units of FauI in NEBuffer 1 (New England Biolabs) for 2 hours at 55° C.

As previously discussed, one of the major factors in determining the efficiency of the siRNA-mediated gene silencing is the selection of target sequences. It is known that not all sequences of a gene are capable of generating functional siRNA and generally, fewer than 50% of the sequences of a gene are able to induce gene silencing. Therefore, an siRNA library derived from cDNA offers a good approach to search for sequences that have potential silencing effect. Thus, a cDNA used in the present invention can be a cDNA sequence of a gene of interest, cDNA library of a gene of interest, or any other cDNA library. By way of example and not of limitation, the cDNA can be the cDNA of green fluorescent protein (GFP), cDNA library of GFP, cDNA library of embryonic stem cells, cDNA library of human brain, cDNA library of mouse 10.5 day embryo, cDNA of activated human macrophages, and the like.

cDNA for a gene of interest is prepared from the mRNA of such gene. As is well known in the art, the mRNA is converted to a complementary single-stranded DNA using the process of reverse transcription, which is subsequently converted to double-stranded DNA using a DNA polymerase. A cDNA library is similarly prepared from mRNA molecules, but either from a variety of genes or from different restriction fragments of the same gene. By way of example and not of limitation, the cDNA library can be prepared from a particular cell type (e.g., embryonic stem cell), particular protein or a particular species (e.g., Drosophila, Xenopus, mouse). Furthermore, the cells used for cDNA preparation may be in a particular state (e.g., activated), infected (e.g., with a virus), diseased (e.g., basal cell carcinoma), and the like. Methods of preparing cDNA libraries are well-known in the art. See, for example, Sambrook and Russell, Molecular Cloning: A Laboratory Manual 3d ed. (2001); and Ausubel et al., Current Protocols in Molecular Biology (1994). Furthermore, cDNA can be obtained from a commercial source since a number of cDNA libraries are available commercially.

Once a cDNA library is obtained, it is treated with at least one restriction enzyme which generates 5′ CG overhangs. With cDNA libraries, it is desirable to cleave cDNA with a large number of enzymes in order to create fragments of different lengths and sequences. Thus, in one embodiment, the cDNA is cleaved with at least three enzymes which create 5′ CG overhangs. By way of example, the cDNA is cleaved with FauI, Hinp1 I and HpyCH4IV. In another embodiment, the cDNA is cleaved with at least five enzymes which create 5′ CG overhangs. Following the digestion of cDNA with restriction enzymes which create 5′ CG overhangs, the DNA oligonucleotide comprising the primer described above is ligated to the cDNA containing 5′ CG overhangs. The ligation reaction is standard in the art and can be performed using e.g., a T4 DNA ligase. Briefly, the DNA oligonucleotide and cDNA are incubated overnight with T4 DNA ligase and a buffer containing ATP at a temperature of about 4° C.

In one embodiment, the DNA oligonucleotide used for the ligation reaction comprises a primer encoding a sequence complementary to the 9 nucleotides at the 3′ end of the H1 promoter. In another embodiment, the DNA oligonucleotide is cleaved with FauI in order to create 5′ CG overhang. In still another embodiment, the DNA oligonucleotide contains about 50 nucleotides.

The ligated molecules are then purified using, e.g., QIAquick kit (Qiagen). For full ligation and DNA purification protocols, see e.g., Sambrook and Russell, Molecular Cloning: A Laboratory Manual 3d ed. (2001). Any of the commercially available ligation kits, such as the Fast-Link™ DNA Ligation kit (EPICENTRE, Madison, Wis.) can also be used. A skilled artisan can readily modify the ligation reaction as is known in the art.

Following the ligation of cDNA with the DNA oligonucleotide, the cDNA is treated with MmeI, which creates cDNA fragments of uniform length, all of which contain the DNA oligonucleotide. The digestion with MmeI is performed, e.g., as follows: ligated DNA is digested in NEBuffer4 with 10 units of MmeI for 2 hours at 37° C. The digestion with MmeI can readily be modified by a skilled artisan depending on a number of factors, such as the amount of cDNA. For example, it may be useful to increase the amount of MmeI used for digestion or to increase the incubation time. Adjusting the conditions for a restriction digestion reaction is standard in the art.

Next, the MmeI digested DNA is ligated with an adaptor oligonucleotide. Similarly as described for the first ligation reaction, the ligation of the MmeI digested DNA to an adaptor oligonucleotide can be carried out using either T4 DNA ligase or a commercial ligation kit, such as, e.g., the Fast-Link™ DNA Ligation kit. If using the T4 DNA ligase, the reaction can generally be carried out at 4° C. overnight.

The adaptor oligonucleotide is an oligonucleotide capable of forming a double-stranded stem (referred to as “stem” only) and a loop, wherein the two strands which form the stem are separated by the loop. In one embodiment, the adaptor oligonucleotide contains from about 19 to about 29 nucleotides. In another embodiment, the oligonucleotide contains about 20 nucleotides. Generally, the stem contains from about 6 to about 10 nucleotides per strand of the stem (i.e. a total from about 12 to about 20 nucleotides), and the loop contains from about 4 to about 11 nucleotides. In one embodiment, the stem contains 6 nucleotides per strand, and the loop contains 9 nucleotides.

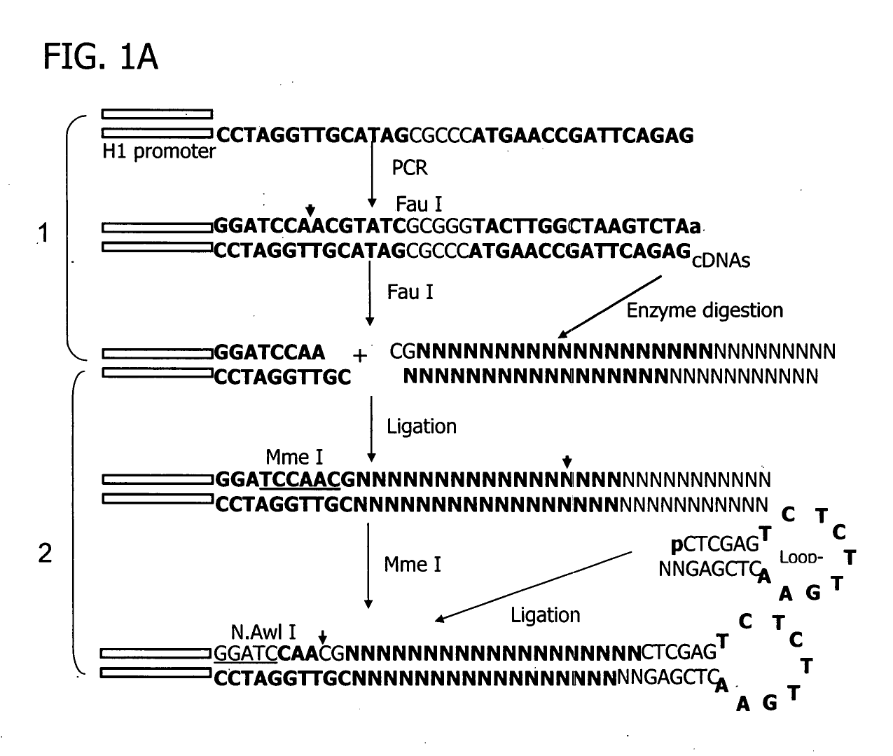

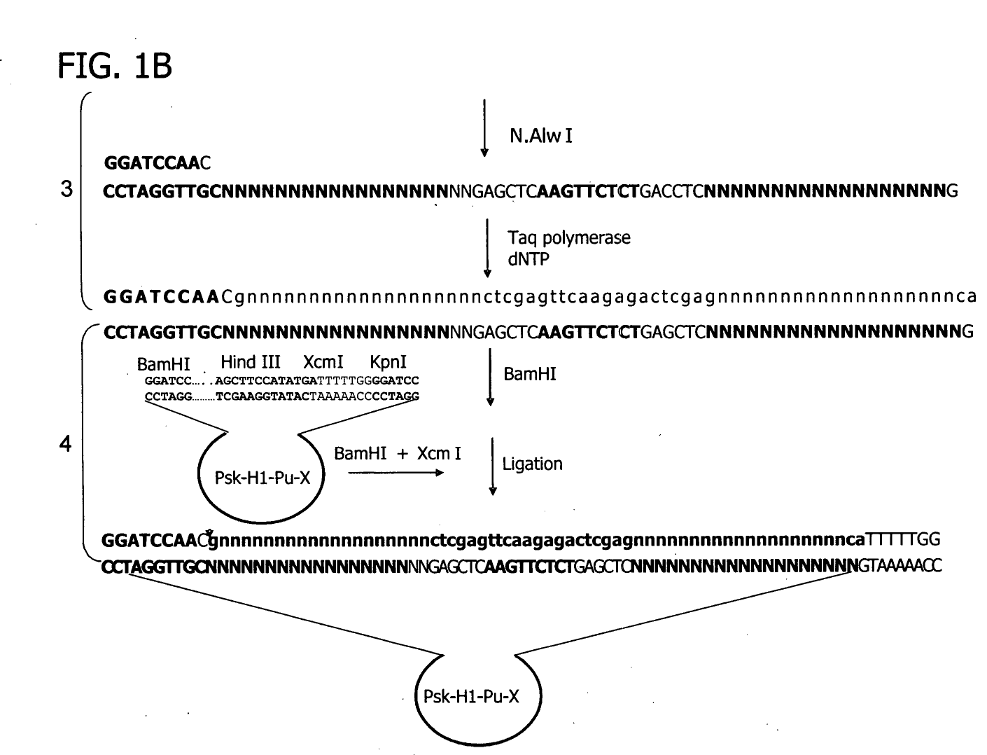

The stem-loop RNA can be designed for a particular application or known stem-loop RNAs, such as siRNA-loop-6, can be used. In one embodiment, the adaptor oligonucleotide is a siRNA-loop-6, as depicted in FIG. 1 with a sequence pCTCGAGTCTCTTGAACTCGAGNN (SEQ ID NO 1). In addition, algorithms exist which will perform stem-loop searches in unaligned RNA sequences. See, for example, Stem-Loop Align SearcH (SLASH), available at http://www.bioinf.au.dk/slash/. Additional algorithms can also be used. See, for example, Gorodkin et al., (Nucleic Acids Research, 29(10):2135-2144 (2001)), who reported that two algorithms FOLDALIGN and COVE, can be used together to discover and model stem-loop RNA motifs in unaligned RNA sequences.

In one embodiment, in order to allow for efficient ligation of the MmeI digested DNA to the adaptor oligonucleotide, the 5′ end of the adaptor oligonucleotide is phosphorylated. Phosphorylation of the 5′ DNA molecule is well known in the art. For example, the phosphorylation can be performed by polynucleotide kinases in the presence of ATP. Furthermore, two degenerate nucleotides are added to the 3′ end of the adaptor oligonucleotide to make the 3′ end compatible for ligation with the MmeI digested DNA. The two degenerate nucleotides can be added during synthesis of the adaptor oligonucleotide. Adding nucleotides as described above is standard in the art, and can be readily performed by a skilled artisan.

Following the ligation of MmeI digested DNA with the adaptor oligonucleotide, the ligated MmeI digested products can be purified. The purification can be performed using a commercially available kit, such as, e.g., QIAquick kit. Alternatively, the DNA can be purified using an agarose gel. Briefly, the ligated DNA is run on an agarose gel, and the desired band is excised from the ethidium-stained gel viewed with a UV transilluminator. Next, the DNA is purified using electroelution, binding and elution from glass or silica particles, or electroelution onto DEAE-cellulose membranes. Purification of DNA can readily be performed by one of ordinary skill in the art.

Next, the ligated MmeI digested products are treated with the nicking enzyme N.Alw I to form a nicked product. Once it binds to its recognition site (5′-GGATCNNNˆN-3′), N.Alw I makes a single cut 4 base pairs away from the recognition site on the top strand. The reaction is carried out at about 37° C. for about two hours; however, a skilled artisan can readily determine other suitable conditions for a particular DNA being treated with N.Alw I. Following the nicking, N.Alw I is either inactivated or preferably removed from the nicked DNA. The nicking enzyme can be inactivated by exposing the nicked product to an elevated temperature as described below. Removal of the nicking enzyme can be performed, e.g., by purifying the nicked DNA. The DNA can be purified using a commercially available DNA purification kit such as QIAquick or ethanol precipitation. In ethanol precipitation, the DNA is centrifuged to form a pellet and separated from the supernatant. The supernatant is next discarded, and the DNA is dissolved in water or an appropriate buffer, such as TE buffer. One of ordinary skill in the art can readily perform these purification steps.

Following the optional purification, the nicked DNA is exposed to an elevated temperature. At the elevated temperature, thermodynamics favor the opening of the N.Alw I treated DNA sequence downstream from the nick, which becomes single stranded while the rest of the sequence remains double stranded. As stated above, the nicking enzyme is also inactivated at the elevated temperature if the nicking enzyme is not removed from the nicked product before the temperature is elevated. In one embodiment, the elevated temperature is from about 65° C. to about 80° C. In another embodiment, the temperature is at least 70° C. In still another embodiment, the temperature is 72° C. At this temperature, the single-stranded palindromic sequence is converted to a double stranded palindromic DNA sequence using a DNA polymerase. It will be appreciated that a skilled artisan can readily determine the most appropriate temperature for opening up a single stranded structure and performing a polymerase reaction. Briefly, this can be determined, e.g., by incubating a single stranded palindromic DNA with a DNA polymerase at different temperatures following the N.Alw I treatment, leaving all the other conditions the same. Next, the double-stranded palindromic DNA obtained in this manner is run on an agarose gel to determine which reaction (i.e. which temperature setting) produced the most double-stranded DNA to identify the preferred temperature.

The treatment with N.Alw I by which the DNA becomes partially single-stranded and partially double-stranded is what allows for the generation of a palindromic structure. Thus, the DNA that is single-stranded as a result of the N.Alw I nicking becomes a palindromic structure. The DNA polymerase used to generate a double stranded palindromic DNA sequence is selected from Taq DNA polymerase, Bst polymerase, and high fidelity polymerases such as Pfu. In one embodiment, the DNA polymerase is Taq DNA polymerase. It is of note that this polymerase reaction does not require primers since the DNA polymerase can use the existing strand in the double stranded region as a primer.

The newly created double stranded palindromic DNA sequence can then be digested with BamH1 for cloning into an expression cassette and/or a vector. The reaction with BamH1 can be performed as is standard in the art. Briefly, the purified DNA is digested in NEBuffer 2 with 5 units of BamH1 for about two hours at about 37° C. The digested double stranded palindromic sequence is next purified, e.g., by agarose gel or any other suitable method known in the art. The digested double-stranded palindromic sequence is a short hairpin DNA at this stage, which will be used for RNAi in a host cell. Once in the cell, shRNA produced by the expression system of this type has a stem-loop structure in which the linker portion forms a loop, and sense and antisense RNAs on its sides pair up to form a stem structure. Then, the loop portion in this palindrome is cleaved by intracellular enzymes to produce the siRNA. siRNA in turn directs the destruction of the homologous mRNA target.

The construction of expression cassettes and/or vectors suitable for practicing the present invention utilizes methods known to those skilled in the art of molecular biology. Accordingly, in one embodiment, the present invention provides a method for expressing short hairpin RNA in a host cell, the method comprising transfecting the host cell with a vector comprising the short hairpin RNA produced by the method described above.

A vector that is used depends on the type of cell which will be used for shRNA expression. The shRNA may be incorporated in a vector that is capable of self-replication in host cells. As one of ordinary skill in the art would recognize, a large variety of such vectors are suitable for use in connection with the present invention. Certain types of vectors allow the expression cassettes to be amplified. Other types of vectors are necessary for efficient introduction of the expression cassettes to cells and their stable expression once introduced. Any vector capable of accepting a DNA expression cassette of the present invention is contemplated as a suitable recombinant vector for the purposes of the invention. The vector may be any circular or linear length of DNA that either integrates into the host genome or is maintained in episomal form. In one embodiment, the vector is a plasmid vector. In another embodiment, the plasmid vector is pSK-H1-Pu-X. Vectors may require additional manipulation or particular conditions to be efficiently introduced into a host cell (e.g., many expression plasmids), or can be part of a self-integrating, cell specific system, such as a recombinant virus. In general, the expression cassettes may be ligated into a DNA transfer vector, such as a plasmid, bacteriophage DNA, or lentiviral, adenoviral, alphaviral, or other viral vector. Exemplary mammalian viral vector systems include adenoviral vectors, adeno-associated type 1 (“AAV-1”) or adeno-associated type 2 (“AAV-2”) viral vectors, hepatitis delta vectors, live, attenuated delta viruses, herpes viral vectors, alphaviral vectors, or retroviral vectors (including lentiviral vectors).

Prokaryotic or eukaryotic host cells may then be transfected or transduced with an appropriate vector such that the shRNA is transcribed in the host cells. The shRNA expression cassettes can also be delivered directly to the host cells by transfection without prior ligation into a DNA transfer vector (e.g., see Castanotto, D. et al., RNA 8: 1454-1460 (2002)).

Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells can be used so long as it successfully introduces at least the shRNA construct into the host cell. These procedures include the use of viral transduction, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra).

The methods of the present invention are applicable to the field of reverse genetic analysis, e.g., by gene silencing. Thus, the present invention provides a method for suppressing expression of a gene of interest by transfecting a host cell with a vector comprising a double stranded palindromic DNA sequence as described above, wherein said sequence forms the short hairpin RNA in the cell and targets the gene of interest. In these methods, the cell is transfected transiently or stably. The effect of the lack of or disappearance of an expressed gene product in the transfected cell can then be assessed, leading to elucidation of the gene's function.

The target gene may be any gene of which it is desired to inhibit or modulate its function. More specifically, the target of shRNA, which is processed into siRNA in the host cell may be an endogenous gene, an exogenous gene (such as a viral gene, pathogenic gene or transfected gene) or a gene of unknown function. The target gene can be, e.g., a developmentally important gene, or it can encode a cytokine, lymphokine, a growth or differentiation factor, a neurotransmitter, an oncogene, a tumor suppressor gene, a membrane channel, or component thereof. The target gene can be one involved in apoptosis. Those of skill in the art will recognize that the above list is not exhaustive. In one embodiment, the target gene will be a gene whose function is unknown. In another embodiment, the target gene will be one associated with a disease or disorder, wherein the cellular expression of shRNA that targets the mRNA of said gene may be used to treat, prevent, or ameliorate that disease or disorder.

The purpose of the gene suppression by RNAi may be therapeutic, prophylactic, or to study the function of a particular gene. The inhibition of the gene may be to alter the phenotype of a cell or organism in some desired way. Typically, the target gene will be a eukaryotic gene, but alternatively the target gene may a viral gene being expressed in a eukaryotic host cell. The target gene may encode a polypeptide or alternatively a structural or enzymatic RNA. However, preferably the target gene encodes a polypeptide.

In another embodiment, the present invention provides a method of identifying at least one gene associated with a selected phenotype, the method comprising the steps of:

(a) transfecting a host cell which exhibits the selected phenotype with a library of expression vectors encoding double stranded palindromic DNAs produced by the method described above, wherein the double stranded palindromic sequence form short hairpin RNAs in a host cell;

(b) identifying, in the host cell which has lost the expression of the selected phenotype, the at least one gene whose expression is suppressed by the short hairpin RNA, wherein the at least one gene so identified is associated with the selected phenotype. By way of example, if a particular host cell exhibits activation in the presence of a pathogen, it may be desirable to determine which gene or genes are responsible for such phenotype. Accordingly, the activated host cells can be transfected with a library of shRNAs produced as described above, and the loss of phenotype (i.e., activation) can be monitored. By suppressing a gene(s) responsible for activation with at least one shRNA, the cell(s) in which such shRNA(s) is expressed will lose the activation status, and can be used to identify the gene(s) associated with activation.

Alternatively, when using a library of shRNAs, it may be desirable to identify particular shRNAs which have the ability to suppress genes in a host cell. Accordingly, the present invention provides a method for identifying potential short interfering RNA sequences, the method comprising inserting into a host cell a vector comprising a double stranded palindromic sequence produced by the method as described herein, and determining which gene exhibits suppressed expression. Additionally, it may be desirable to identify shRNAs which can suppress a particular gene to a certain degree (e.g., 50% or 90% suppression). Thus, in both methods, a library of shRNAs is transfected into a host cell, and then the host cell is assayed for gene suppression.

The suppression of the gene can be measured in a variety of ways, typically at the RNA, protein or phenotypic level. In one embodiment, the level of gene expression may also be determined at the protein level. Various immunological assays are routinely used by those skilled in the art to measure the level of a gene product, particularly using polyclonal or monoclonal antibodies that react specifically with a protein product. In addition, functional assays may also be performed to confirm the suppressed expression of one or more genes in transfected/transduced cells. Depending on the particular gene family and the known biological functions the gene products normally exert, specific assays can be designed for detecting a decreased level of activity. For example, when the targeted gene family encodes enzymes, specific enzymatic assays can be carried out using suitable substrates to detect the enzymatic activity in the transfected or transduced cells. When the targeted genes encode kinases, for instance, the lack of kinase activity in transfected/transduced cells may be reflected in reduced level of phosphorylation of the substrates; when the targeted genes encode receptors, such as cytokine receptors, the diminished gene expression may be reflected in reduced response to the ligands; when the targeted genes encode tumor suppressors or oncogenes, the decreased gene expression may be reflected in changes, e.g., in the tumorigenic tendency and/or metastatic potential of the transfected or transduced cells. Other possible changes in phenotypes that can indicate the reduced gene expression include: viral susceptibility—HIV infection; autoimmunity—inactivation of lymphocytes; drug sensitivity—drug toxicity and efficacy; graft rejection—MHC antigen presentation, etc.

Suppression can be confirmed using biochemical techniques such as Northern blotting, nuclease protection, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), in situ hybridization, immunoprecipitation, enzyme function, as well as phenotypic assays known to those of skill in the art. To examine the extent of gene silencing, samples or assays of the organism of interest or cells in culture expressing a particular shRNA are compared to control samples lacking expression of the shRNA. Control samples (lacking construct expression) are assigned a relative value of 100%. Inhibition of expression of a target gene is achieved when the test value relative to the control is about 90%, preferably 50%, more preferably 25-0%.

Suppression in a cell line or whole organism, may be measured by using a reporter or drug resistance gene whose protein product is easily assayed. Such reporter genes and selection markers include any of those mentioned herein. Inhibition may also be measured at the phenotypic level. For example, the appearance of a phenotype similar to that associated with disruption of the targeted gene can be observed. Where the purpose of the siRNA is to block expression of a gene associated with a disease, whether or not the disease is prevented, ameliorated or treatable using the siRNA can be measured. Where the purpose of the siRNA is to treat an infectious disease, any reduction in viral or bacterial load can be assessed or alternatively the presence, absence or severity of symptoms associated with the disorder can be measured.

In general terms, these techniques involve transfecting or transducing a population of cells with the siRNA expression library and monitoring the population of cells for any phenotypic change, such as decrease or increase in expression of mRNA, proliferation, differentiation, apoptosis, or senescence, etc. For example, an siRNA library targeting the tyrosine kinase family can be used to identify tyrosine kinases that function in the normal apoptotic pathway as follows. The library is delivered to a population of cells by transduction with a retroviral vector. The transduced cells are then subjected to a stimulus that induces apoptosis in normal cells (e.g., treatment with etoposide, cisplatin, or ionizing radiation). The majority of the treated cells will die due to this treatment. However, if a tyrosine kinase participates in the apototic pathway downstream of the stimulus, then cells expressing an siRNA against this tyrosine kinase will survive due to the siRNA-mediated defect in the apoptotic pathway. SiRNA expression cassettes are rescued from the surviving cells by PCR or other methods known to those skilled in the art. Putative tyrosine kinases that function in the apoptotic pathway are then identified from the siRNA sequences.

In addition to the protocol details provided above and in the examples, the information regarding PCR reactions, ligation reactions, restriction digestion, DNA purification, cell transformation and the like can be found in the literature. Basic texts disclosing these general methods for use in connection with this invention include Sambrook and Russell, Molecular Cloning: A Laboratory Manual 3d ed. (2001); and Ausubel et al., Current Protocols in Molecular Biology (1994).

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

Example 1

Oligonucleotides were synthesized through commercial sources (Sigma Genosys, Woodlands, Tex., and IDT, Coralville, Iowa). DNA sequencing service was provided by Northwoods DNA (Solway, Minn.). Restriction enzymes were obtained from New England Biolabs (Beverly, Mass.). Taq polymerase was purchased from ABI (Foster City, Calif.).

A ˜700 bp PCR fragment from the GFP coding region was amplified using primers GFP-5.1A and GFP-3.1. PCR was performed at the following conditions: 1 cycle of 94° C. for 3 min, 30 cycles of 94° C. for 0.5 min, 52° C. for 1 min and 72° C. for 0.5 min, followed by extension at 72° C. for 10 min. The primers used were GFP-5.1A (sense), 5′-ATGGTGAGCAAGGGCGAGGAGCT (SEQ ID NO 2) and GFP-3.1 (antisense), 5′-AGTACTTGTACAGCTCGTCCAT (SEQ ID NO 3). After purification, the PCR product was digested with restriction enzymes Hinp1 I and HpyCH4 IV, both of which generate CG overhanging at 5′ end. 5 μg DNA was digested with 5 U each of Hinp1 I and HpyCH4 IV at 37° C. for 2h. Agarose gel analysis revealed multiple bands, as predicted. After purification, the digested cDNAs were ready to be ligated to the H1 promoter.

To amplify the H1 promoter, PCR reactions were carried out using H1-5.1 and H1-MmeI-siRNA-3.1 and resulted in a single band of ˜130 bp. The PCR product adaptor carrying the H1 promoter was relatively large so it was not necessary to separate them by polyacrylamide gel electrophoresis (PAGE). Following digestion of 5 μg PCR product with FauI in NEBuffer 1 (New England Biolabs) for 2 h at 55° C., a ˜110 bp fragment was detected. This FauI-digested product was then ligated to GFP cDNA with 5′ CG overhanging. The ligation was performed at 4° C. overnight with 2 μg DNA and 200 U of T4 DNA ligase (New England Biolabs). After ligation and MmeI digestion (in NEBuffer 4 with 10 U MmeI for 2 h at 37° C.), a DNA band with ˜130 bp was detected and subsequently purified for the next round of ligation. Since siRNA loop-6 is relatively small, the ligation reaction was carried out at 4° C. A typical ligation reaction for siRNA-loop-6 was performed using Fast link ligation kit (Epicentre, Madison, Wis.) at 4° C. overnight. The targeted ligated DNA was separated from the rest of the ligated products by an agarose gel and then purified using gel purification kit (Qiagen). Under such a condition, a band around ˜145 bp was detected, suggesting that the DNA fragments were successfully ligated to siRNA-loop-6. Two percent agarose gel was used for all experiments.

After nicking with N.Alw I (for 1 μg DNA, 10 U N.Alw I was used for 2 h at 37° C.), the nicked DNA was purified by a DNA purification kit (QIAquick). Alternatively, an ethanol precipitation method can be used as is known in the art. The purified product was extended by 2.5 U of Taq polymerase in the presence of 0.2 mM dNTP at 72° C. for 10 min, followed by digestion with BamH1 (in NEBuffer 2 with 5 U BamH1 for 2 h at 37° C.). Finally, the product was cloned into Pu-H 1-pSK-X that was cut with BamH1 and Xcm I.

To determine whether siRNAs generated using this method can inhibit GFP expression, Pu-H1-pSK-X was introduced into 293T cells that were also co-transfected with pEGFP-C3 (Clontech). 293T cells were purchased from American Tissue Culture Collection (ATCC, Manassas, Va.) and were grown in DMEM medium (Cambrex, East Rutherford, N.J.). All media were supplemented with 10% FBS, 2 mM L-glutamine, 100 units of penicillin/ml and 100 μg streptomycin/ml (Cambrex). Cells were incubated at 37° C. in a humidified chamber supplemented with 5% CO2.

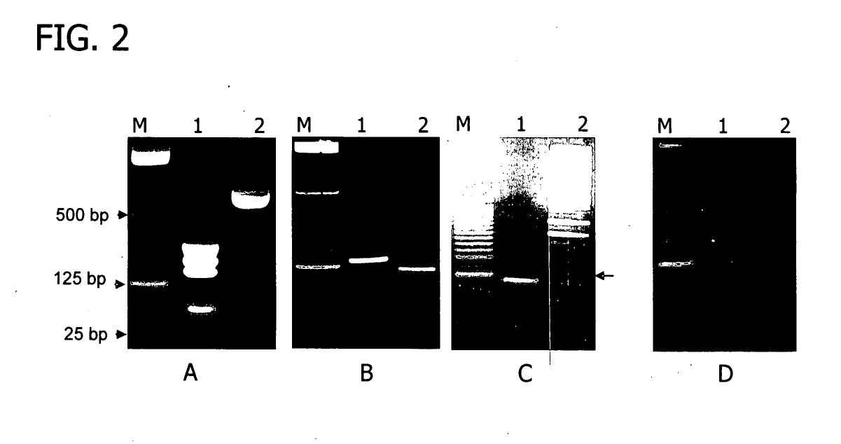

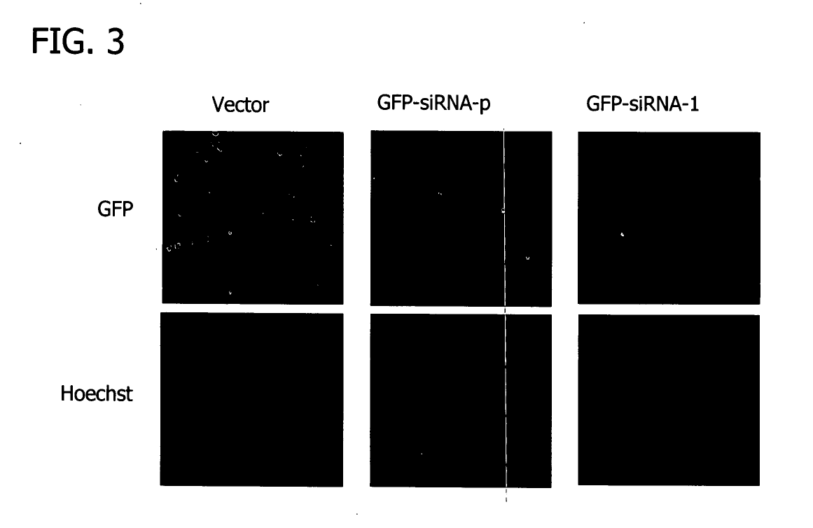

Plasmid DNAs carrying an appropriate sequence were introduced into 293T cells by calcium phosphate method as described previously by Mo et al. (J Biol Chem 277:2958-64, 2002). Cells were examined under microscope 48 h after transfection. To examine the expression of GFP level, cells were trypsinized and transferred to a cell culture cover slip in 6-well plates. Before the examination, cell nuclei were stained with Hoechst dye (Sigma, St. Louis, Mo.) at 10 μg/ml for 10 min at room temperature. As a positive control, the GFP sequence 5′-GAAGAAGTCGTGCTGCTTC (SEQ ID NO 4) was cloned into pSUPER as GFP-siRNA-p and used in transfection experiments. A result for one clone (GFP-siRNA-1) is shown in FIG. 2. DNA sequence for GFP-siRNA-1 was 5′-GTCTATATCATGGCCGACA (SEQ ID NO 5), corresponding to nucleotides 450-468. Compared to vector control, this clone caused over 50% suppression of GFP expression, which was comparable to the positive control.

What is claimed is:

1. A method for preparing a double stranded palindromic DNA sequence which forms a short hairpin RNA in a host cell, the method comprising:
a) obtaining a DNA oligonucleotide comprising a primer encoding a sequence which is complementary to the 3′ end of a promoter, a 5′ CG overhang, an MmeI recognition site, a restriction enzyme recognition site containing GGATCC, and an N.Alw I recognition site, wherein the MmeI recognition site overlaps the restriction enzyme recognition site, and the N.Alw I recognition site overlaps the restriction enzyme recognition site;
b) obtaining cDNA of a gene of interest or a cDNA library, wherein the cDNA contains a 5′ CG overhang;
c) ligating the DNA oligonucleotide with the cDNA to form a ligated product;
d) digesting the ligated product with MmeI to form an MmeI-digested product;
e) ligating the MmeI-digested product with an adaptor oligonucleotide to form a ligated MmeI-digested product, wherein the adaptor oligonucleotide forms a double-stranded stem and a loop;
f) treating the ligated MmeI-digested product with a nicking enzyme N.Alw I to form a nicked product;
g) removing or inactivating the nicking enzyme from the nicked product;
h) exposing the nicked product to an elevated temperature such that the nicked product becomes single-stranded downstream from a nick site and remains double-stranded upstream from a nick site, thereby generating a single-stranded palindromic sequence; and
i) generating a double-stranded palindromic DNA sequence from the single-stranded palindromic DNA sequence.

2. The method of claim 1, further comprising digesting the double stranded palindromic sequence with a restriction enzyme whose recognition site contains GGATCC.

3. The method of claim 1, wherein the restriction enzyme is BamH1.

4. The method of claim 1, wherein the promoter is selected from the group consisting of CMV promoter, a CAGGS promoter, U6 promoter, H1 promoter, a tRNA promoter, a 7SL RNA promoter and a 5 S rRNA promoter.

5. The method of claim 4, wherein the promoter is H1 promoter.

6. The method of claim 1, wherein the 5′ CG overhang is created by a restriction enzyme selected from the group of FauI, HinpI, Hinp1I, BsaHI, AciI, HpalI, HpyCHIV, HpyCH41V, TaqaI, Acl I, DspD I, Cla I, Msp I, Nar I, and BstB I.

7. The method of claim 1, wherein the DNA oligonucleotide contains from about 40 to about 70 nucleotides.

8. The method of claim 7, wherein the DNA oligonucleotide contains from about 50 to about 60 nucleotides.

9. The method of claim 8, wherein the DNA oligonucleotide contains about 54 nucleotides.

10. The method of claim 7, wherein the primer sequence contains from about 9 to about 30 nucleotides.

11. The method of claim 1, wherein the adaptor oligonucleotide contains from about 19 to about 29 nucleotides.

12. The method of claim 11, wherein the loop of the adaptor oligonucleotide contains from about 4 to about 11 nucleotides.

13. The method of claim 11, wherein the stem of the adaptor oligonucleotide contains from about 6 to about 10 nucleotides.

14. The method of claim 11, wherein the adaptor oligonucleotide contains 20 nucleotides.

15. The method of claim 1, wherein the digestion with the nicking enzyme is performed at about 37° C.

16. The method of claim 1, wherein the generation of a double stranded palindromic DNA sequence is performed at a temperature of at least 70° C.

17. The method of claim 1, wherein the double-stranded palindromic DNA sequence is generated by a DNA polymerase.

18. The method of claim 17, wherein the DNA polymerase is selected from the group consisting of Taq DNA polymerase and Bst polymerase.

19. The method of claim 2, wherein the digested product is inserted into an expression vector.

20. The method of claim 1, wherein the cDNA library is digested with at least two restriction enzymes creating 5′ CG overhangs.

21. The method of claim 20, wherein the cDNA library is digested with at least five restriction enzymes creating 5′ CG overhangs.

22. A method for preparing a double stranded palindromic DNA sequence which forms a short hairpin RNA in a host cell, the method comprising:
a) digesting a DNA oligonucleotide comprising a primer encoding a sequence which is complementary to the 3′ end of H1 promoter with FauI, an MmeI recognition site which overlaps a BamH1 recognition site, a FauI recognition site immediately after the MmeI recognition site, and an N.Alw I recognition site which overlaps the BamH1 recognition site;
b) digesting cDNA of a gene of interest or cDNA library with at least one enzyme which creates a 5′ CG overhang;
c) ligating the primer containing DNA fragment with the digested cDNA to form a ligated product;
d) digesting the ligated product with MmeI to form a MmeI-digested product;
e) ligating the MmeI-digested product with an adaptor oligonucleotide to form a ligated MmeI-digested product, wherein the adaptor oligonucleotide forms a double-stranded stem and a loop;
f) treating the ligated MmeI-digested product with a nicking enzyme N.Alw I to form a nicked product;
g) removing or inactivating the nicking enzyme from the nicked product;
h) exposing the nicked product to an elevated temperature such that the nicked product becomes single-stranded downstream from a nick site and remains double-stranded upstream from the nick site, thereby generating a single-stranded palindromic sequence;
i) generating a double-stranded palindromic DNA sequence from the single-stranded palindromic DNA sequence;
j) digesting the double-stranded palindromic DNA sequence with BamH1 to form a BamH1-digested product; and
k) inserting the BamH1-digested sequence into an expression vector.

23. A method for expressing short hairpin RNA in a host cell, the method comprising transfecting the host cell with a vector comprising a double stranded palindromic DNA sequence produced by the method of claim 1.

24. A method for suppressing expression of a gene of interest by transfecting a host cell with a vector comprising a double stranded palindromic DNA sequence produced by the method of claim 1, wherein the short hairpin RNA targets the gene of interest.

25. A method for identifying potential short interfering RNA sequences, the method comprising inserting into a host cell a vector comprising a double stranded palindromic DNA sequence produced by the method of claim 1, determining which gene exhibits suppressed expression, and identifying a short interfering RNA sequence which targets said gene.

26. A method of identifying at least one gene associated with a selected phenotype, the method comprising the steps of:
(a) transfecting a host cell which exhibits the selected phenotype with a library of expression vectors encoding double stranded palindromic DNA sequences produced by the method of claim 1, wherein the double stranded palindromic DNA sequences form short hairpin RNA sequences in the host cell; and
(b) identifying, in the host cell which has lost the expression of the selected phenotype, the at least one gene whose expression is suppressed by the short hairpin RNA, wherein the at least one gene so identified is associated with the selected phenotype.


SEQUENCE LISTING (5 sequences)

SEQ ID NO: 1 - DNA, 23 bp, Artificial Sequence
  siRNA-loop-6
  misc_feature (1)..(1): n = pC (phosphorylated C)
  misc_feature (22)..(23): n = any nucleotide
ntcgagtctcttgaactcgagnn

SEQ ID NO: 2 - DNA, 23 bp, Artificial Sequence
  Primer
atggtgagcaagggcgaggagct

SEQ ID NO: 3 - DNA, 22 bp, Artificial Sequence
  Primer
agtacttgtacagctcgtccat

SEQ ID NO: 4 - DNA, 19 bp, Artificial Sequence
  GFP-siRNA-p
gaagaagtcgtgctgcttc

SEQ ID NO: 5 - DNA, 19 bp, Artificial Sequence
  GFP-siRNA-1
gtctatatcatggccgaca